(19) United States
(12) Patent Application Publication
(10) Pub. No.: US 2009/0080607 A1
(43) Pub. Date: Mar. 26, 2009

(54) DEVICE AND PROCESS FOR FAT ANALYSIS

(76) Inventors: Hartmut Hoffmann, Bad Laasphe (DE); Stefan Linn, Runkel (DE)

Correspondence Address: MCNEES WALLACE & NURICK LLC, 100 PINE STREET, P.O. BOX 1166, HARRISBURG, PA, 17108-1166, US

(21) Appl. No.: 12/092,698

(22) PCT Filed: Oct. 20, 2006
(86) PCT No.: PCT/EP06/10138

§ 371 Date: Sep. 22, 2008

(30) Foreign Application Priority Data
Nov. 7, 2005 (DE) 10 2005 053 348.5

(51) Int. Cl. G01N 23/083; B02C 18/30; G01N 33/12; G01P 3/00; G01G 11/00; G01N 21/89; B02C 18/36

(52) U.S. Cl. 378/53; 241/82.1; 241/82.4; 73/493; 73/433

(57) ABSTRACT

Meat processing device having at least one comminution unit for comminution and/or mixing of fresh and/or frozen meat and having at least one transport means (2) on which the meat can be transported and which is arranged, in the direction of processing, downstream of the comminution unit (1), which has a fat analysis device (3) for determining the fat content, the density in the meat and/or the BEFFE (connective tissue protein free meat protein), and also process for fat analysis.

The present invention relates to a meat processing device having at least one comminution unit for comminuting and/or mixing fresh and/or frozen meat and having at least one conveying means, on which the meat can be conveyed and which is arranged after the comminution unit in the processing direction and which has a fat analysis device for determining the fat content, the density in the meat and/or the BEFFE (connective tissue protein free meat protein), and a process for fat analysis.

Meat products must or should not exceed a specific fat content, therefore accurate fat analysis of meat and an exact setting of specific fat contents in meat products play an increasing role. The analysis of the fat content of the meat is preferably carried out during the processing in a meat processing device. There are therefore a series of proposals as to how these measurements are to be carried out but which all have the disadvantage that they are very complicated and/or to some extent inaccurate or are carried out only very late in the process.

The fat analysis can already be carried out continuously, for example by the fat content of meat on a conveyor belt being determined by a sensor. However, these measurements have the disadvantage that the mass flow of the meat must be absolutely constant.

There is therefore the object of providing a device and a process for the continuous determination of the fat content of meat which do not have the disadvantages of the prior art.

According to the invention, the object is achieved by a meat processing device having at least one comminution unit for comminuting and/or mixing fresh and/or frozen meat and having at least one conveying means, on which the meat can be conveyed and which is arranged after the comminution unit in the processing direction, a fat analysis device for the continuous determination of a fat content of the meat being arranged in the region of the comminution unit, and by a weighing device being arranged on the conveying means, it being possible for the weight of the meat conveyed on the conveying means to be determined continuously by the weighing device.

With the meat processing device according to the invention, the fat content of fresh meat, frozen meat and/or their mixture can be analyzed. It was entirely surprising to those skilled in the art and not to be expected that, with the meat processing device according to the invention, it is possible to determine very accurately the fat content of the meat product to be processed.

The device according to the invention has the advantage that the fat content of meat can be determined continuously, it being possible for the mass flow of the meat likewise to be determined continuously or at least semi-continuously by the weighing device and not having to be constant. The device can be produced simply and integrated simply into a meat production process. In particular, it is possible to retrofit the fat analysis device and/or the weighing device in existing meat processing lines. The measurement accuracy is about ±1%, based on the standard deviation.

A meat processing device in the sense of the invention is any machine known to those skilled in the art for meat processing, with which meat is comminuted and/or mixed. However, the meat processing device is preferably a mixer or a comminution machine, in particular a mincer, or a combination thereof.

Suitable conveying means are any conveying devices suitable for processing foodstuffs. In a preferred embodiment, the conveying means is a conveyor belt, preferably a motor-driven plastic conveyor belt, on which the meat is transported away from the comminution unit, for example, preferably a mincer, for example into a container or mixer.

The weighing device in the device according to the invention is preferably a belt weigher, which can advantageously easily be integrated into the conveyor belt. Particularly preferably, the weighing device has a measuring section, it being possible for an instantaneous weight per unit area of the meat on the measuring section to be determined by the weighing device either semi-continuously or continuously.

A suitable fat analysis means is any fat analysis means familiar to those skilled in the art. However, the fat analysis means preferably has a radiation source, for example having a plurality of energy stages and a radiation detector. In a particularly preferred embodiment, the radiation source is an x-ray source and the radiation detector is an x-ray detector. Likewise preferred are an infrared source as a radiation source and an infrared detector as a radiation detector.

In the case of fat analysis by means of an x-ray detector, the attenuation of the x-ray beam is preferably measured in an energy range between 18 and 45 keV. The measurement path, in particular the layer of meat through which radiation passes, is preferably 20 to 300 mm, particularly preferably 50 to 100 mm, quite particularly preferably 50-70 mm. The calculation of the fat content and the control of the x-ray source are carried out by a microprocessor or programmable logic controller (PLC).

The fat analysis is likewise preferably carried out with near infrared transmission (NIT). The fat analysis can be carried out at any point of the comminution unit in which a measuring section between the radiation source and the radiation detector is at least for some time not interrupted by moving parts, in particular metal parts.

The comminution unit preferably has at least one conveying unit, for example a screw conveyor, the conveying unit forcing the meat through the comminution unit. Meat in the sense of the invention is any product which at least partly contains meat. The fat analysis can be carried out, for example, in the region of the conveying unit.

It is further preferred for the comminution unit to have at least one pre-cutter and, in addition, for example a perforated disk which interacts with a knife. In the pre-cutter or in the perforated disk and/or the knife, the meat is comminuted and/or mixed. The fat analysis device is preferably arranged in a pre-cutter, what is known as a measuring pre-cutter. The latter can be arranged after a further pre-cutter in relation to the processing direction of the meat. This measuring pre-cutter has apertures, at the edges of which, following the pre-comminution, the meat is preferably not comminuted further in the further precutter. The measuring section of the fat analysis device is preferably arranged in one of these apertures.

The additional pre-cutter before the measuring precutter has the advantage that wear takes place on the pre-cutter and, as a result, is at least reduced on the measuring pre-cutter. As a result of the pre-comminution of the product in the pre-cutter, the accuracy of the fat analysis is increased. For instance, the pre-cutter can also interact with an additional knife, which is arranged before the precutter in relation to the processing direction of the meat. This additional knife ensures a clean cut of the meat, so that, for example, no connective tissue accumulates in the region of the pre-cutter. This prevents meat with any desired fat content remaining in the measuring chamber for a relatively long time, which likewise has a positive effect on the quality of analysis.

In a preferred embodiment, the meat processing device according to the invention additionally has a speed measurement, so that, besides the instantaneous fat content and the instantaneous weight, the instantaneous speed of the meat can also be determined, it not being necessary for the measurement of the conveying speed to be carried out in the region of the fat analysis device or the weighing device. The measurement of the instantaneous conveying speed of the meat is preferably carried out by an optical measuring method and likewise preferably following the comminution and/or mixing of the meat, virtually with no pressure, which is to say 30 approximately at ambient pressure. The optical measuring method is based, for example, on a light source, such as a halogen lamp, combined with a CCD camera.

The conveying speed of the meat likewise preferably corresponds to the belt speed of the conveying means, which can preferably be determined by counting the belt revolutions. In this way, a complicated measurement of the speed can advantageously be dispensed with. Particularly preferably, the fat analysis device and the weighing device operate continuously. The measurements can be evaluated as a function of time or as a function of belt revolutions. For instance, the evaluation is carried out every one to two seconds or every 5 to 10 cm of the belt length of the conveying means.

A further subject of the present invention is a process for the continuous determination of the fat content of meat with a device described previously, the fat content of the meat being determined continuously with the fat analysis device and the weight of the meat being determined continuously with the weighing device and, by incorporating a distance between the weighing device and the fat analysis device and also the conveying speed of the meat, an instantaneous fat content of a mass flow of the meat being calculated.

Those skilled in the art will understand that the meat of which the fat content is measured instantaneously is weighed only after a time delay. The time delay in turn depends on the distance between the measuring points and the conveying speed, the distance generally being constant while the speed can likewise be constant or predefined or preferably can be measured.

The time-dependent mass flow Ft is preferably calculated in accordance with the following formula:

Ft [g/s]=G [g/cm²]*b [cm]*v [cm/s]

where:
G is the instantaneous weight per unit area in the measuring section (5),
b is the width of the measuring section (5) and
v is the conveying speed of the meat.

The measurements can likewise preferably be carried out as a function of the belt revolution of the conveying means. In this case, instead of a time dependence of the mass flow, a dependence on the belt revolutions is given.

The mass flow Fb in gram per revolution, based on the belt revolutions of the conveying means, is calculated in accordance with the following formula

Fb [g/rev]=G [g/cm²]*b [cm]*1 [cm/rev]

where:
G is the instantaneous weight per unit area in the measuring section (5),
b is the width of the measuring section (5) and
1 is the belt length of the conveying means (2) in cm per revolution.

Particularly preferably, an average fat content of a resultant meat mixture is calculated from the instantaneous fat content f of the meat and its mass flow F, in particular by the products of instantaneous fat content f and associated mass flow Ft or Fb being summed and divided by the summed mass flow, in accordance with the formula:

Fat content=ΣFt*f/ΣFt=ΣFb*f/ΣFb.

Those skilled in the art will understand that the sum ΣFt is made by summing over time and the sum ΣFb is made by summing over the belt revolutions of the conveying means. This method has the advantage that, as a result of the continuous calculation of the average fat content of a meat mixture, a time delay is advantageously avoided. It is not necessary for any additional devices to be provided with which the average fat content of the meat mixture is determined intermediately or after finishing. The fat content can be determined both in fresh and in frozen meat. The consistency of the meat is not changed by the process according to the invention.

The values of the fat content and the weight values of the meat can be averaged over a time period of, for example, 1 to 10 seconds, preferably 2 to 4 seconds, or an appropriate belt advance of the conveying means.

With the process according to the invention, it is in particular possible to also measure the fat content in the event of a non-constant conveying rate. With the process according to the invention, it is possible to assign the instantaneous fat content to the associated mass flow of the meat examined and transfer this data for example to a central computer, so that, for example, a maximum fat content of the product can be indicated on the product to be sold.

A further subject of the invention is a process for setting a fat content in a meat mixture by using a meat processing device described previously, it being possible for at least two conveying streams which differ in their fat content to be applied to the meat processing device, an actual fat content of the resultant meat mixture being determined continuously in accordance with the process described previously, an intended fat content being specified and, in the event of an intended/actual deviation of the fat content, a mixing ratio of the conveying streams being changed.

This process according to the invention has the advantage that the fat content in a meat mixture can be adjusted very accurately and specifically. According to the invention, it is possible to determine the fat content of the meat mixture accurately at any time and, consequently, to correct the fat content of the meat yet to be conveyed into the meat mixture. As a result of the continuous ability to influence the fat content of the meat mixture, the result is a saving in time for the manufacturer.

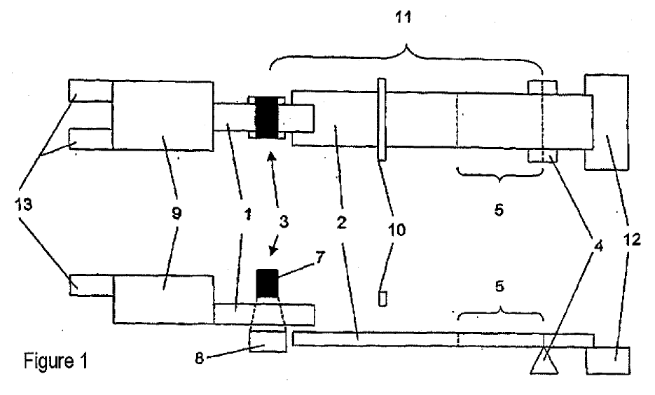

In the following text, the invention will be explained by using FIGS. 1-3. These explanations apply both to the device according to the invention and to the process according to the invention. They are merely exemplary and do not restrict the general idea of the invention.

FIG. 1 shows a schematic illustration of a meat processing device according to the invention.

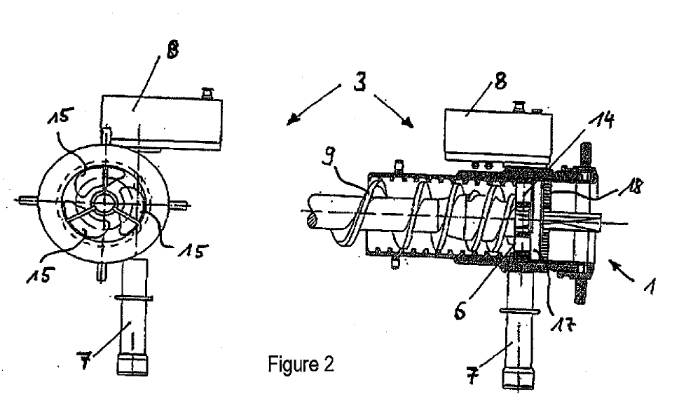

FIG. 2 shows an exemplary embodiment of a comminution unit with fat analysis device.

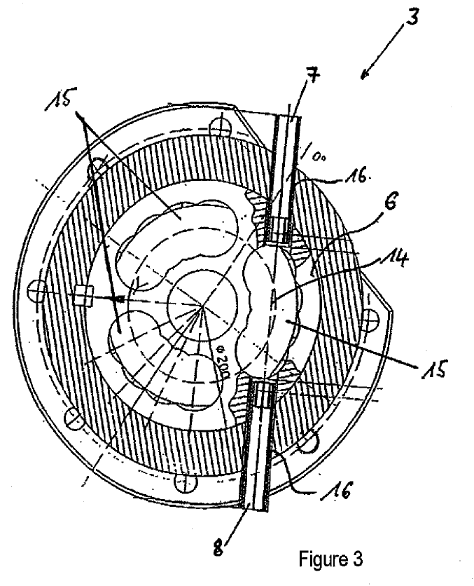

FIG. 3 shows the arrangement of the fat analysis device in the region of a measuring pre-cutter in detail.

In FIG. 1, the meat processing device according to the invention is illustrated schematically in two views. The meat processing device has a comminution unit 1 and a conveying unit 9 which, in the sense of this invention, is defined as part of the comminution unit 1. Via at least two conveying streams 13, which are illustrated beside each other in the top view, the meat processing device is fed with fresh and/or frozen meat. The latter is comminuted and/or mixed in the comminution unit.

The meat from the conveying streams 13 has different proportions of fat, for example. By means of regulating the conveying streams 13, an instantaneous fat content of the mass flow through the meat processing device can thus be influenced and, as a result, an average fat content can be adjusted specifically in the resultant meat mixture 12. The transport of the comminuted and/or mixed meat from the comminution unit 1 to a container having the meat mixture 12 is carried out by a conveying means 2, preferably a belt conveyor. The conveying speed of the meat is measured continuously by a speed measurement 10.

Arranged in the region of the comminution unit 1 is a fat analysis device 3, which has a radiation source 8 and a radiation detector 7, which can be seen best in the lower illustration. For example, this is an x-ray source 7 and an x-ray detector 8. The meat is forced through the comminution unit 1 past the x-ray source 8 by the conveying unit 9 and, in the process, x-rays pass through it. The x-ray detector 7 measures the absorption of the x-rays by the meat. By using this information, conclusions can be drawn about the fat content of the meat.

Arranged on the conveying means 2 is a weighing device 4, preferably a belt weigher. By using the weighing device 4, the weight of the meat which is located on a measuring section 5 of the conveyor belt 2 can be determined. Those skilled in the art will see that from these continuously determinedly measured values, taking into account the conveying speed, a mass flow of the meat through the meat processing device according to the invention can be calculated.

The fat analysis device 3 and the weighing device 4 are 5 spaced apart from each other at a distance 11 along the conveying section. As a result of the physical separation of the devices, simpler measuring instruments can be used. Integration into existing processing lines is simple and inexpensive. Those skilled in the art will understand that the measured values from the fat analysis device 3 and the weighing device 4 must be assigned to one another. The assignment depends on the time interval which elapses while an arbitrarily small portion of meat of which the fat content has just been determined is transported to the weighing device 4. This can easily be determined from the distance 11 and the conveying speed. The conveying speed is preferably determined by the speed measurement 10 or via the belt revolution speed of the conveying means 2.

By summing the products of mass flow values and associated fat contents and subsequently dividing by the summed mass flow, that is to say the current total weight of the meat mixture 12, the current fat content of the meat mixture 12 is obtained.

FIG. 2 shows a comminution device 1 with a fat analysis device 3 in two views. A conveying unit 9, here a screw, conveys the meat through a cutter set of the comminution device 1. The cutter set comprises a pre-cutter 6, a knife 17 arranged downstream and a perforated disk 18, the knife 17 interacting with the perforated disk 18. Those skilled in the art will see that there can be a further pre-cutter and likewise a further knife before the pre-cutter 6. The wear on the pre-cutter 6 would be reduced to a great extent.

In the region of the measuring pre-cutter 6, the measurement is carried out by the fat analysis device 3, which comprises a radiation source 7 and a radiation 5 detector 7. A beam, here an x-ray beam 14, is emitted by the radiation source 8 and, as can be seen from the left-hand part of FIG. 2, passes through the aperture 15 in the measuring pre-cutter 6 and, at the end of the aperture 15, is received by the radiation detector 7 and analyzed. Those skilled in the art will see that other measuring principles can also be used. In the case of fat analysis devices in the region of moving parts, it is important that the measuring section is not interrupted at the time of the measurement or, should the measuring section be interrupted at the time of measurement, that these measuring values are discarded.

FIG. 3 shows a detailed illustration of the measuring pre-cutter 6. The pre-cutter 6 has three or more apertures 15, through which the meat is forced. In the region of one of the apertures 15 there are arranged holes 16, into which the radiation source 8 and the radiation detector 7 are respectively inserted. The radiation source 8 emits an x-ray beam 14, which passes through the meat which is located in one of the apertures 15. On account of the different absorption of the radiation 14 by fat and lean meat, the fat content of the meat located in the aperture can be determined.

### LIST OF DESIGNATIONS

1 Comminution unit
2 Conveying means
3 Fat analysis device
4 Weighing device
5 Measuring section
6 Pre-cutter
7 Radiation detector
8 Radiation source
9 Conveying unit
10 Speed measurement
11 Distance
12 Meat mixture
13 Conveying stream
14 Measuring beam
15 Aperture
16 Hole
17 Knife
18 Perforated disk

1. A meat processing device having at least one comminution unit for comminuting and/or mixing fresh and/or frozen meat and having at least one conveying means, on which the meat can be conveyed at a determinable conveying speed and which is arranged after the comminution unit in the processing direction, a fat analysis device for the continuous determination of a fat content of the meat being arranged in the comminution unit, characterized in that a weighing device is arranged on the conveying means, it being possible for the weight of the meat conveyed on the conveying means to be determined continuously by the weighing device and, by incorporating a distance between the weighing device and the fat analysis unit and also the conveying speed of the meat, it being possible to assign an instantaneous fat content of the meat to a mass flow.

2. The meat processing device as claimed in claim 1, characterized in that the conveying means is a conveyor belt, preferably a motor-driven plastic conveyor belt.

3. The meat processing device as claimed in claim 1, characterized in that the weighing device is a belt weigher.

4. The meat processing device as claimed in claim 1, characterized in that the weighing device has a measuring section, it being possible for an instantaneous weight per unit area of the measuring section to be determined continuously by the weighing device.

5. The meat processing device as claimed in claim 1, characterized in that the fat analysis devices has a radiation source and a radiation detector, and in that the fat analysis device is preferably based on X-radiation and/or near infrared transmission (NIT).

6. The meat processing device as claimed in claim 1, characterized in that the comminution unit has at least one conveying unit, the conveying unit forcing the meat through the comminution unit.

7. The meat processing device as claimed in claim 1, characterized in that the comminution unit has a pre-cutter and the fat analysis device is arranged in the precutter.

8. The meat processing device as claimed in claim 1, characterized in that it has a speed measurement for determining a conveying speed of the meat, the speed measurement preferably being carried out by an optical method.

9. The meat processing device as claimed in claim 1, characterized in that the speed measurement is carried out virtually without pressure after the comminution and/or mixing.

10. The meat processing device as claimed in claim 1, characterized in that the conveying speed of the meat corresponds to the belt speed of the conveying means, which can preferably be determined by counting the belt revolutions.

11. The meat processing device as claimed in claim 1, characterized in that the fat analysis devices and the weighing device operate continuously, it preferably being possible for the measurements to be evaluated as a function of time or as a function of belt revolutions.

12. A process for the continuous determination of the fat content of meat with a device as claimed in claim 1, characterized in that the fat content of the meat is determined continuously with the fat analysis device and in that the weight of the meat is determined continuously with the weighing device and in that, by incorporating a distance between the weighing device and the fat analysis device and also the conveying speed of the meat, an instantaneous fat content is assigned to a mass flow of the meat.

13. The process as claimed in claim 12, characterized in that the mass flow Ft over time is calculated in accordance with the following formula:
Ft [g/s]=G [g/cm²]*b [cm]*v [cm/s]
where:
G is the instantaneous weight per unit area in the measuring section,
b is the width of the measuring sections and
v is the conveying speed of the meat.

14. The process as claimed in claim 12, characterized in that the mass flow Fb in gram per revolution, based on the belt revolutions of the conveying means (2), is calculated in accordance with the following formula:
Fb [g/rev]=G [g/cm²]*b [cm]*I [cm/rev]
where:
G is the instantaneous weight per unit area in the measuring sections,
b is the width of the measuring section and
I is the belt length of the conveying means in cm per revolution.

15. The process as claimed in claim 12, characterized in that an average fat content of a resultant meat mixture is calculated from the instantaneous fat content f of the meat and its mass flow Ft of Fb, in particular by the products of instantaneous fat content f and associated mass flows F being summed and divided by the summed mass flow, in accordance with the formula:
Fat content=ΣFt*f/ΣFt=ΣFb*f/ΣFb,
the summation ΣFt being made over time and the summation ΣFb being made over the revolutions of the conveying means.

16. (canceled)
